(19) United States
(12) Patent Application Publication
(10) Pub. No.: US 2021/0251296 A1
(43) Pub. Date: Aug. 19, 2021

(54) AEROSOL GENERATION DEVICES

(71) Applicant: OMEGA LIFE SCIENCE LTD., Migdal Haemeq (IL)

(72) Inventor: Miron HAZANI, Haifa (IL)

(21) Appl. No.: 17/253,592

(22) PCT Filed: Jun. 24, 2019
(86) PCT No.: PCT/IL2019/050701

§ 371 Date: Dec. 17, 2020

Related U.S. Application Data
(60) Provisional application No. 62/689,841, filed on Jun. 26, 2018.

(51) Int. Cl. A24F 40/51; A24F 40/485; A24F 40/53

(52) U.S. Cl. CPC A24F 40/51; A24F 40/53; A24F 40/485

(57) ABSTRACT

The present disclosure generally relates to the field of aerosol generation device.

### TECHNICAL FIELD

The present disclosure generally relates to the field of aerosol generation devices.

### BACKGROUND

Cigarettes impart superb user experience when smoked owing partially to the smoker's ability to self-titrate the dose of inhaled nicotine. In other words, puff intensity and duration play a crucial role in determining the actual dose of nicotine inhaled, and subsequently delivered to the smoker.

Current endeavors to reduce the inherent risk associated with smoking are manifested by the introduction of new categories of products classified as reduced-risk-products, claimed to lower the inherent risk associated with smoking. Numerous available product types, such as e-cigarettes and heat-not-burn Tobacco products, attempt to reduce harmful effects of cigarette smoking. Such attempts, while may be considered partially effective, still exhibit several drawbacks. One particular drawback stems from the fact that such products lack a viable option for self-titration, as opposed to combustible cigarettes. While some product types allow varying the degree of nicotine, which is directly proportional with puff duration, i.e. the time allotted by a user for a discrete puff (inhalation) from the device, the overall effect is marginal at best. Moreover, although reduced risk products potentially carry a smaller risk than that associated with conventional cigarettes, they still contain excipients such as propylene glycol (PG) and vegetable glycerin (VG), the clinical effects and potential risks of which remain controversial.

WO 2016/059630 to the inventor of the present invention discloses a nebulizer comprising a porous medium configured to produce aerosols, a displaceable wetting mechanism configured to spread a liquid over the porous medium thereby to wet the porous medium and a gas channel configured to introduce pressure gradient to the porous medium.

There is an unmet need for a device that upon inhalation, generates aerosol in which the dosages of the aerosolized substance are determined by the length and intensity of a user's inhalation.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other advantages or improvements.

According to one aspect, there is provided an aerosol generating device, comprising at least one porous medium, a gas inlet, a proximal compartment between the gas inlet and the at least one porous medium, an outlet, a distal compartment between the at least one porous medium and the outlet, a first pressure sensor configured to detect the pressure in the distal compartment and to generate signals indicative thereof, a gas pump configured to deliver compressed gas via the gas inlet, through the proximal compartment, towards the at least one porous medium, and a Central Processing Unit (CPU) configured to receive signals from the first pressure sensor, and configured to control operation of the gas pump.

According to some embodiments, there is provided an aerosol generating device comprising: at least one porous medium; a gas inlet; a proximal compartment between the gas inlet and the at least one porous medium; an outlet; a distal compartment between the at least one porous medium and the outlet; a first pressure sensor configured to detect the pressure in the distal compartment and to generate first pressure signals indicative thereof; a second pressure sensor configured to detect the pressure in the proximal compartment and to generate second pressure signals indicative thereof; a gas pump configured to deliver compressed gas via the gas inlet, through the proximal compartment, towards the at least one porous medium; a liquid pump configured to deliver liquid from a liquid reservoir to the at least one porous media, through a liquid conduit; and a Central Processing Unit (CPU) configured to receive the first pressure signals from the first pressure sensor and the second pressure signals from the second pressure sensor, wherein the CPU is further configured to control operation of the gas pump in response to the first pressure signals; and configured to control operation of the liquid pump in response to the first pressure signals and the second pressure signals.

According to some embodiments, the aerosol generating device further comprises a first Pulse Wave Modulation component, controlled by the CPU and configured to adjust the flow rate of gas from the gas pump.

According to some embodiments, the aerosol generating device further comprises a power source compartment configured to house at least one power source.

According to some embodiments, the power source compartment comprises a power source configured to power the gas pump, the liquid pump, or both.

According to some embodiments, the aerosol generating device further comprises a measurement conduit, open at one end thereof to the distal compartment, and connected at the other end thereof to the first pressure sensor.

According to some embodiments, the first pressure sensor comprises a first differential pressure sensor.

According to some embodiments, the first pressure sensor is configured to measure sub atmospheric pressure. According to some embodiments, the first pressure sensor is configured to measure atmospheric and sub atmospheric pressure. According to some embodiments, the first pressure sensor is configured to measure pressure in the range of 0.01 to 1 Bar.

According to some embodiments, the CPU is configured to variably control operation of the gas pump as a function of the first pressure signals.

According to some embodiments, the CPU is configured to detect whether pressure measured by the first pressure sensor is higher or lower than a threshold pressure value, and is configured to activate the gas pump while the pressure detected by the first pressure sensor is lower than the pressure threshold value.

According to some embodiments, the CPU is further configured to determine whether the pressure in the distal compartment is higher or lower than a threshold pressure value, based on said first pressure signals, and is configured to activate the gas pump when said pressure is lower than the pressure threshold value.

According to some embodiments, the CPU is further configured to deactivate the gas pump when said pressure is higher than the pressure threshold value.

According to some embodiments, the CPU is configured to vary the power supplied to the gas pump, wherein the gas pump is configured to deliver compressed gas at variable compression levels in response to the varying power supplied thereto.

According to some embodiments, the CPU is configured to vary the power supplied to the gas pump based on the first pressure signals.

According to some embodiments, the CPU is configured to increase the power supplied to the gas pump upon decrease of the pressure in the distal compartment.

According to some embodiments, the CPU is configured to analyze the rate of change of the pressure measured by the first pressure sensor.

According to some embodiments, the CPU is configured to variably control operation of the gas pump as a function of the signals received from the first pressure sensor.

According to some embodiments, the porous medium comprises a liquid.

According to some embodiments, the liquid comprises a nicotine formulation.

According to some According to some embodiments, the liquid comprises an aqueous nicotine formulation.

According to some embodiments, the aerosol generating device further comprises a second pressure sensor configured to detect the pressure in the proximal compartment and to generate signals indicative thereof, wherein the CPU is further configured to receive signals from the second pressure sensor.

According to some embodiments, the second pressure sensor comprises a second differential pressure sensor.

According to some embodiments, the second pressure sensor is configured to measure super atmospheric pressure. According to some embodiments, the second pressure sensor is configured to measure atmospheric and super atmospheric pressure. According to some embodiments, the second pressure sensor is configured to measure pressure in the range of 1 to 100 Bar.

According to some embodiments, the aerosol generating device further comprises a liquid pump configured to deliver liquid from a liquid reservoir to the at least one porous media, through a liquid conduit.

According to some embodiments, the CPU is further configured to variably control operation of the liquid pump.

According to some embodiments, the CPU is configured to variably control operation of the liquid pump as a function of the second pressure sensor.

According to some embodiments, the CPU is configured to variably control operation of the liquid pump as a function of the first pressure signals.

According to some embodiments, the CPU is further configured to determine whether the pressure in the distal compartment is higher or lower than a threshold pressure value, based on said first pressure signals, and is configured to activate the liquid pump when said pressure is lower than the pressure threshold value.

According to some embodiments, the CPU is further configured to deactivate the liquid pump when said pressure is higher than the pressure threshold value.

According to some embodiments, controlling the operation of the liquid pump in response to the first pressure signals comprises controlling the rate of liquid delivery by the liquid pump to the at least one porous medium based on the first pressure signals.

the CPU is configured to increase the rate of liquid delivery to the porous medium upon decrease of the pressure in the distal compartment.

According to some embodiments, the CPU is configured to variably control operation of the liquid pump as a function of the second pressure signals.

According to some embodiments, the CPU is further configured to determine whether the pressure in the proximal compartment is higher or lower than a threshold pressure value, based on said second pressure signals, and is configured to activate the liquid pump when said pressure is lower than the pressure threshold value.

According to some embodiments, the CPU is further configured to deactivate the liquid pump when said pressure is higher than the pressure threshold value.

According to some embodiments, controlling the operation of the liquid pump in response to the second pressure signals comprises controlling the rate of liquid delivery by the liquid pump to the at least one porous medium based on the second pressure signals.

According to some embodiments, the CPU is configured to increase the rate of liquid delivery to the porous medium upon decrease of the pressure in the proximal compartment.

According to some embodiments, the CPU is configured to analyze the rate of change of the pressure measured by the second pressure sensor.

According to some embodiments, the aerosol generating device further comprises a second Pulse Wave Modulation component, controlled by the CPU and configured to adjust the flow rate of liquid from the liquid pump.

According to another aspect, there is provided an aerosol generating device, comprising at least one proximal porous medium, at least one distal porous medium, a gas inlet, a proximal compartment between the gas inlet and the at least one porous medium, an outlet and a distal compartment between the at least one porous medium and the outlet, wherein the proximal porous medium is in contact with the distal porous medium, and wherein the proximal porous medium is characterized with higher porosity than the distal porous medium.

According to some embodiments, the proximal porous medium comprises a plurality of pores having first average diameter and the distal porous medium comprises a plurality of pores having second average diameter, wherein the ratio between the first average diameter and the second average diameter is at least 10:1, at least 5:1 or at least 2:1.

According to some embodiments, the ratio between the number of pores of the proximal porous medium and the number of pores of the proximal porous medium is at least 2:1, at least 4:1, at least 5:1, at least 10:1 or at least 20:1.

According to some embodiments, the aerosol generating device further comprises a first pressure sensor configured to detect the pressure in the distal compartment and to generate signals indicative thereof, a gas pump configured to deliver compressed gas via the gas inlet, through the proximal compartment, towards the at least one porous medium, and a CPU configured to receive signals from the first pressure sensor, and configured to control operation of the gas pump.

According to some embodiments, there is provided an aerosol generating device comprising: at least one proximal porous medium; at least one distal porous medium; a gas inlet; a proximal compartment between the gas inlet and the at least one proximal porous medium; an outlet; a distal compartment between the at least one distal porous medium and the outlet; a first pressure sensor configured to detect the pressure in the distal compartment and to generate first pressure signals indicative thereof; a gas pump configured to deliver compressed gas via the gas inlet, through the proximal compartment, towards the at least one proximal porous medium; and a Central Processing Unit (CPU) configured to receive the first pressure signals from the first pressure sensor and configured to control operation of the gas pump in response to the first pressure signals. wherein the proximal porous medium is in contact with the distal porous medium; and wherein the proximal porous medium is characterized with higher porosity than the distal porous medium.

According to some embodiments, the proximal porous medium comprises a plurality of pores having first average diameter and the distal porous medium comprises a plurality of pores having second average diameter, wherein the ratio between the first average diameter and the second average diameter is at least 2:1, at least 6:1 or at least 10:1. According to some embodiments, the ratio between the number of pores of the proximal porous medium and the number of pores of the proximal porous medium is at least 2:1, at least 3:1, at least 6:1, at least 10:1 or at least 15:1.

According to some embodiments, the aerosol generating device further comprises a filter configured to filter the passage of droplets depending on their diameter, such that large diameter droplets are obstructed thereby; wherein the filter is in contact with the distal porous medium

According to some embodiments, the aerosol generating device further comprises a liquid container and a liquid drawing element dipped within the liquid container, configured to deliver liquid from the liquid container towards the at least one distal porous medium

According to some embodiments, the aerosol generating device further comprises a first Pulse Wave Modulation component, configured to be controlled by the CPU and configured to adjust the flow rate of gas from the gas pump.

According to some embodiments, the CPU is configured to control operation of the gas pump as a function of the signals received from the first pressure sensor.

According to some embodiments, the liquid drawing element is in contact with the at least one distal porous medium

According to another aspect, there is provided an aerosol generating device, comprising at least one porous medium, a gas inlet, a proximal compartment between the gas inlet and the at least one porous medium, an outlet, a distal compartment between the at least one porous medium and the outlet, a liquid container and a liquid drawing element dipped within the liquid container, configured to deliver liquid from the liquid container towards the at least one porous medium.

According to some embodiments, the liquid drawing element is in contact with the at least one porous medium.

According to some embodiments, the aerosol generating device further comprises a liquid-gating porous medium, wherein the liquid-gating porous medium is in contact with the at least one porous medium, wherein the liquid drawing element is in contact with the liquid-gating porous medium, and wherein the liquid-gating porous medium is characterized by higher porosity than the at least one porous medium and lager pores than the pores of the at least one porous medium.

According to some embodiments, the aerosol generating device further comprises a first pressure sensor configured to detect the pressure in the distal compartment and to generate signals indicative thereof, a gas pump configured to deliver compressed gas via the gas inlet, through the proximal compartment, towards the at least one porous medium, and a CPU configured to receive signals from the first pressure sensor, and configured to control operation of the gas pump.

According to some embodiments, the aerosol generating device further comprises a first Pulse Wave Modulation component, configured to be controlled by the CPU and configured to adjust the flow rate of gas from the gas pump.

According to some embodiments, the CPU is configured to control operation of the gas pump as a function of the signals received from the first pressure sensor

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1 constitutes a schematic illustration of an aerosol generating device, according to some embodiments;

FIG. 2 depicts graphs of pressure and gas pump output as functions of time, according to some embodiments;

FIG. 3 constitutes a schematic partial illustration of aerosol generating device, according to some embodiments;

FIG. 4 constitutes a schematic partial illustration of aerosol generating device, according to some embodiments;

FIG. 5A depicts matric potential vs. liquid saturation functions, according to some embodiments;

FIG. 5B depicts matric potential vs. liquid saturation functions, according to some embodiments;

FIG. 5C depicts matric potential vs. liquid saturation functions, according to some embodiments;

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

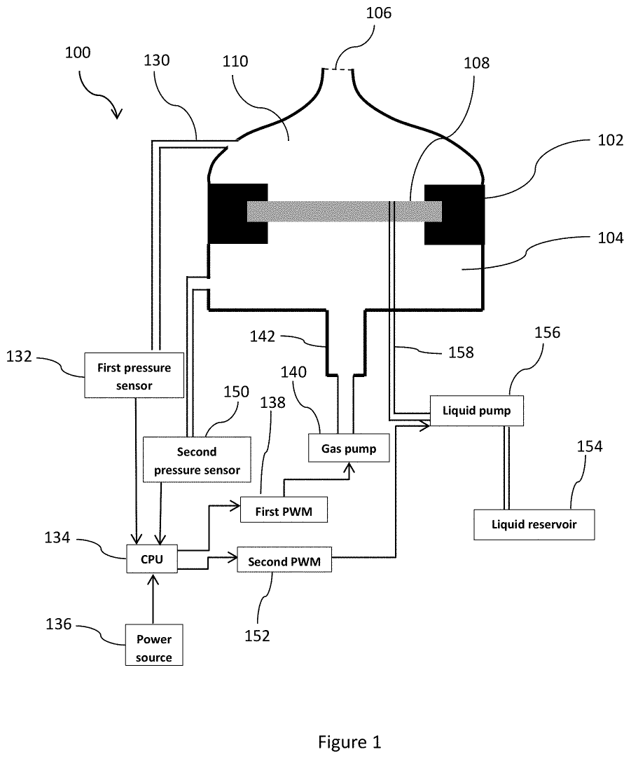

Reference is now made to FIG. 1. FIG. 1 constitutes a schematic illustration of an aerosol generating device 100, according to some embodiments. Aerosol generating device 100 comprises at least one porous medium 108, a proximal compartment 104, a gas inlet 142, a distal compartment 110 and an outlet 106.

Within the context of this specification the term “proximal” generally refers to the side or end of any device or a component of a device, which is closer to the gas inlet 142.

Within the context of this specification the term “distal” generally refers to the side or end of any device or a component of a device, which is opposite the “proximal end”, and is closer to outlet 106.

According to some embodiments, aerosol generating device 100 is an inhaler or a nebulizer. According to some embodiments, aerosol generating device 100 is an inhaler. According to some embodiments, aerosol generating device 100 is a nebulizer.

A porous medium is understood to be a two-phase product with voids and solid portions. Generally, in an open cell porous mediums the voids are interconnected, and the solid portions, which define the voids, are also interconnected. As a result, such structures have a plurality of pores where inner surfaces of individual pores may be accessible from neighboring pores. In contrast, in closed cell porous mediums individual pores are separate and self-contained.

As used herein, the term “porous” refers to any material that includes one or more of pores, cracks, fissures, vugs and voids extending into the material from external surfaces thereof. Further, the term “pore” includes and encompasses cracks, fissures, vugs and voids. Porous materials may include, for example, sponge, felt, paper, sand, cotton-wool silica, concrete, alumino-silicates, metals, minerals, polymers, ceramics, composites, asphalt and brick. Typically, the pores allow a fluid flow therethrough, including liquid materials, such as aqueous solutions.

The term ‘porous medium’ as used herein refers to any material that is capable of incorporating, taking in, drawing in or soaking liquids, and upon applying physical pressure thereto, release a portion or the entire amount/volume of the absorbed liquid. The physical pressure may be achieved for example by pressing the material against a solid structure.

According to some embodiments, the at least one porous medium 108 is a sponge, a tissue, a foam material, a fabric, a porous metal or any other material capable of fully or partially retrievably absorbing liquids. Each possibility is a separate embodiment of the invention.

According to some embodiments, the at least one porous medium 108 is rigid. According to some embodiments, the at least one porous medium 108 is made of metal. According to some embodiments, the at least one porous medium 108 has two flat sides, which remain flat when liquid is pressed there through. According to some embodiments, the at least one porous medium 108 is rigid where liquid is absorbed, or partially absorbed, therein.

The term “partially absorbed” and “partially saturated”, as used herein, are interchangeable and refer to the percentage of liquid absorbed in the pores of the porous material, wherein 0% refers to a porous material where all of its pores are vacant of liquid. Thus, the term “partially absorbed ” may refer to a porous material wherein at least 0.005% of the pores contain liquid, or wherein the overall contents of the vacant space within the porous material occupied with liquid is 0.005%. According to some embodiments, partially absorbed refers to at least 0.001% liquid contents within the porous material. According to some embodiments, partially absorbed refers to at least 0.05% liquid contents within the porous material. According to some embodiments, partially absorbed refers to at least 0.01% liquid contents within the porous material. According to some embodiments, partially absorbed refers to at least 0.5% liquid contents within the porous material. According to some embodiments, partially absorbed refers to at least 0.1% liquid contents within the porous material. According to some embodiments, partially absorbed refers to at least 1% liquid contents within the porous material. According to some embodiments, partially absorbed refers to at least 5% liquid contents within the porous material. According to some embodiments, partially absorbed refers to at least 10% liquid contents within the porous material. According to some embodiments, partially absorbed refers to at least 20% liquid contents within the porous material. According to some embodiments, partially absorbed refers to at least 30% liquid contents within the porous material. According to some embodiments, partially absorbed refers to at least 40% liquid contents within the porous material. According to some embodiments, partially absorbed refers to at least 50% liquid contents within the porous material.

According to some embodiments, the at least one porous medium 108 is configured to enable small diameter droplets to pass through the structure thereof and to obstruct large diameter droplets from passing through the material thereof.

According to some embodiments, aerosol generating device 100 further comprises at least one container configured to contain liquid to be delivered to the at least one porous medium 108. According to some embodiments, the liquid comprises a nicotine formulation. According to some embodiments, the nicotine formulation is an aqueous nicotine formulation.

According to some embodiments, the at least one porous medium 108 is disposable. According to some embodiments, the at least one porous medium 108 is in the form of a rod, a capsule or a flat disc. Each possibility represents a separate embodiment. According to some embodiments, the at least one porous medium 108 is in the form of a flat disc.

As used herein the terms “aerosol” or “aerosolized drug” refer to a suspension of solid or liquid particles in a gas. As used herein “aerosol” or “aerosolized drug” may be used generally to refer to a drug that has been vaporized, nebulized, being in a form of spray or jet or otherwise converted from a solid or liquid form to an inhalable form including suspended solid or liquid drug particles. According to some embodiments, the drug particles include nicotine particles.

According to some embodiments, the at least one porous medium 108 is having two sides, wherein a proximal side is facing proximal compartment 104 and a distal side is facing distal compartment 110.

Without being bound by any theory or mechanism, a pressure gradient at porous medium 108 reflects the presence of value difference between the pressure at the proximal side of porous medium 108 and the pressure at the distal side of porous medium 108, such that pressure values vary inside the volume of porous medium 108. These values range from the pressure value at the proximal side to the pressure value at the distal side of the porous medium.

According to some embodiments, aerosol generating device 100 further comprises a support 102. According to some embodiments, the at least one porous medium 108 is attached to support 102 such that unintentional displacement of the at least one porous medium 108 in the distal or proximal directions is prevented. According to some embodiments, the at least one porous medium 108 is attached to support 102 such that displacement of the at least one porous medium 108 in the distal or proximal directions is avoided.

According to some embodiments, outlet 106 is configured to deliver the aerosols to a respiratory system of a user of aerosol generating device 100. According to some embodiments, outlet 106 is connected to a mouthpiece. According to some embodiments, outlet 106 is mechanically connected to a mouthpiece. According to some embodiments, the mouthpiece is detachable.

According to some embodiments, aerosol generating device 100 is mobile. According to some embodiments, aerosol generating device 100 is portable. According to some embodiments, aerosol generating device 100 is handheld. According to some embodiments, aerosol generating device 100 is powered by a mobile power source.

According to some embodiments, gas inlet 142 is a gas delivery channel configured to introduce pressure gradient to porous medium 108. According to some embodiments, gas inlet 142 is a gas delivery channel configured to introduce pressurized gas to porous medium 108. According to some embodiments, gas inlet 142 is a gas suction channel configured to introduce sub-pressurized gas to porous medium 108.

According to some embodiments, proximal compartment 104 is a pressurized gas container configured to deliver pressurized gas from gas inlet 142 to porous medium 108 and create an over-atmospheric pressure on one side of porous medium 108, thereby induce a pressure gradient at porous medium 108.

The term ‘pressurized gas’ as used herein is interchangeable with the term ‘compressed gas’ and refers to gas under pressure above atmospheric pressure.

According to some embodiments, aerosol generating device 100 further comprises a gas pump 140. According to some embodiments, gas pump 140 is configured to deliver compressed gas to porous medium 108 via gas inlet 142.

According to some embodiments, gas pump 140 is an air pump, gas inlet 142 is an air inlet, and proximal compartment 104 is a pressurized air compartment.

According to some embodiments, the at least one porous medium 108 is filled with liquid intended to be aerosolized. According to some embodiments, the at least one porous medium 108 is partially filled with liquid intended to be aerosolized. When pressurized gas or pressurized air is driven through the at least one porous medium 108, the liquid is drained from at least some of the pores of the at least one porous medium 108 towards the distal side thereof, where aerosolization occurs. When the gas or air stops its flow through the at least one porous medium 108, the aerosolization process stops and liquid from the distal side of the at least one porous medium 108 undergoes imbibition back thereto. In this manner, the at least one porous medium 108 acts both as an atomizing element for aerosolization, and as a liquid reservoir, according to some embodiments.

According to some embodiments, distal compartment 110, bounded between the at least one porous medium 108 and outlet 106, is exposed to ambient pressure such as atmospheric pressure when outlet 106 is openly exposed to the environment, and is exposed to reduced suction pressure exerted thereon by the mouth of a user of aerosol generating device 100 when the user inhales through outlet 106.

According to some embodiments, aerosol generating device 100 further comprises a first pressure sensor 132, configured to detect the pressure in distal compartment 110 and to generate signals indicative thereof. According to some embodiments, the signals are first pressure signals. According to some embodiments, first pressure sensor 132 is configured to detect the pressure distal compartment 110 and to generate first pressure signals indicative thereof. According to some embodiments, first pressure sensor 132 comprises a first differential pressure sensor. According to some embodiments, first pressure sensor 132 is configured to measure sub atmospheric pressure. According to some embodiments, first pressure sensor 132 is configured to measure atmospheric and sub atmospheric pressure. According to some embodiments, first pressure sensor 132 is configured to measure pressure in the range of 0.01 to 1 Bar. According to some embodiments, first pressure sensor 132 is positioned within distal compartment 110. According to some embodiments, first pressure sensor 132 is attached to a sidewall of aerosol generating device 100. According to some embodiments, aerosol generating device 100 further comprises a measurement conduit 130, open at one end thereof to distal compartment 110, and connected at the other end thereof to first pressure sensor 132 (see FIG. 1).

The term “conduit”, as used herein, is interchangeable with any one or more of the terms channel, port, passage, opening, pipe and the like.

According to some embodiments, aerosol generating device 100 further comprises a central processing unit (CPU) 134. According to some embodiments, CPU 134 is configured to receive signals from first pressure sensor 132, indicative of the pressure values measured by first pressure sensor 132. According to some embodiments, CPU 134 is configured to control operation of gas pump 140. According to some embodiments, CPU 134 is configured to control operation of gas pump 140 as a function of the signals received from first pressure sensor 132.

According to some embodiments, CPU 134 is configured to receive the first pressure signals from first pressure sensor 132. According to some embodiments, CPU 134 is further configured to control operation of gas pump 140 in response to the first pressure signals.

According to some embodiments, CPU 134 is configured to control operation of gas pump 140 by changing the voltage supplied thereto. When low voltage is supplied to gas pump 140, it will generate lower gas flow, developing a smaller pressure drop across the at least one porous medium 108, resulting in a smaller amount of liquid being mobilized to the distal side of the at least one porous medium 108 and a lower aerosolization yield. When high voltage is supplied to gas pump 140, it will generate higher gas flow, developing a larger pressure drop across the at least one porous medium 108, resulting in a larger amount of liquid being mobilized to the distal side of the at least one porous medium 108 and a higher aerosolization yield.

According to some embodiments, CPU 134 is configured to control operation of gas pump 140 as a function of the first pressure signals. According to some embodiments, CPU 134 is configured to detect whether pressure measured by first pressure sensor 132 is higher or lower than a threshold pressure value, and is configured to activate gas pump 140 while the pressure detected by the first pressure sensor is lower than the pressure threshold value. According to some embodiments, CPU 134 is further configured to determine whether the pressure in distal compartment 110 is higher or lower than a threshold pressure value, based on the first pressure signals, and is configured to activate gas pump 140 when said pressure is lower than the pressure threshold value. According to some embodiments, CPU 134 is further configured to deactivate gas 140 pump when said pressure is higher than the pressure threshold value.

Distal compartment 110 is located between porous medium 108 and outlet 106, through which an aerosol generating device user inhales. As a result of an inhalation, the pressure inside distal compartment 110 is reduced. First pressure sensor 132 is configured to sense this pressure drop, and in response send corresponding first pressure signals to CPU 134, according to some embodiments. According to some embodiments, upon receiving the first pressure signals indicative of sub atmospheric pressure, CPU 134 activates gas pump 140, which in return generates pressurized gas, through gas inlet 142 towards porous medium 108. Upon the pressurized gas hitting porous medium 108, which contains liquid, an aerosol forms and proceeds though outlet 106 to the user's mouth and respiratory tract according to some embodiments. When the user stops inhaling the pressure inside distal compartment 110 is elevated back to atmospheric pressure. First pressure sensor 132 is configured to sense this pressure drop, and in response send corresponding first pressure signals to CPU 134, according to some embodiments. According to some embodiments, upon receiving the first pressure signals indicative of atmospheric pressure, CPU 134 deactivates gas pump 140, according to some embodiments.

According to some embodiments, the relation between CPU 134, gas pump 140 and first pressure sensor 132 are not limited to on/off operation, but also enable pressure variability. Without wishing to be bound by any theory or mechanism of action, when a nicotine aerosol generating device user inhales deeply and for a prolonged duration, it is indicative that the user requires a high dosage of nicotine. Conversely, when a nicotine aerosol generating device user inhales briefly, it is indicative that the user requires a low dosage of nicotine. A variable operation of aerosol generating device 100 may satisfy the need for such correlation and agreement. Specifically,

As a result of a deep and/or prolonged inhalation, the pressure inside distal compartment 110 is reduced to a low level and/or for a prolonged period of time. First pressure sensor 132 is configured to sense this low pressure level and/or prolonged period of time, and in response send corresponding first pressure signals to CPU 134 to operate at high voltage and/or for a prolonged period of time, according to some embodiments. According to some embodiments, upon receiving the first pressure signals indicative of low pressure level and/or prolonged period of time of low pressure, CPU 134 activates gas pump 140, to operate at high voltage and/or for a prolonged period of time which in return generates a substantial amount of pressurized gas for a prolonged period, through gas inlet 142 towards porous medium 108. Upon the substantial amount of pressurized gas hitting porous medium 108, which contains liquid, a substantial amount of aerosol forms and proceeds though outlet 106 to the user's mouth and respiratory tract according to some embodiments.

Conversely, as a result of a short inhalation, the pressure inside distal compartment 110 is reduced to a moderated level and/or for a short period of time. First pressure sensor 132 is configured to sense this moderate pressure level and/or short period of time, and in response send corresponding first pressure signals to CPU 134 to operate at moderate voltage and/or for a short period of time, according to some embodiments. According to some embodiments, upon receiving the first pressure signals indicative of moderate pressure level and/or short period of time of low pressure, CPU 134 activates gas pump 140, to operate at moderate voltage and/or for a short period of time which in return generates a moderate amount of pressurized gas for a short period, through gas inlet 142 towards porous medium 108. Upon the moderate amount of pressurized gas hitting porous medium 108, which contains liquid, a moderate amount of aerosol forms and proceeds though outlet 106 to the user's mouth and respiratory tract according to some embodiments.

According to some embodiments, CPU 134 is configured to vary the power supplied to gas pump 140. According to some embodiments, gas pump 140 is configured to deliver compressed gas at variable compression levels in response to the varying power supplied thereto. According to some embodiments, gas pump 140 is configured to deliver compressed gas for variable durations in response to the varying power supplied thereto.

According to some embodiments, CPU 134 is configured to vary the power supplied to gas pump 140 based on the first pressure signals.

According to some embodiments, CPU 134 is configured to increase the power supplied to gas pump 140 upon decrease of the pressure in distal compartment 110.

According to some embodiments, CPU 134 is configured to analyze the rate of change of the pressure measured by first pressure sensor 132. According to some embodiments, CPU 134 is configured to vary the power supplied to gas pump 140 based on said analysis.

According to some embodiments, CPU 134 is configured to control operation of gas pump 140 as a function of the signals received from first pressure sensor 132.

According to some embodiments, aerosol generating device 100 further comprises a first Pulse Wave Modulation (PWM) component 138, such that the flow rate of gas or air exiting gas or air pump 140 is adjusted via first PWM component 138 by CPU 134.

According to some embodiments, aerosol generating device 100 further comprises a communication element (not shown) configured to enable wireless communication of aerosol generating device 100 with servers, databases and personal devices (e.g. computers, mobile phones) among others.

According to some embodiments, the communication element provides wireless communication through Bluetooth, WiFi, Zigbee and/or Z-wave.

According to some embodiments, aerosol generating device 100 further comprises a power source compartment 136, configured to house at least one power source, such as a battery. The at least one power source is configured to provide power to at least one of: CPU 134, first pressure sensor 132, first PWM 138 and gas pump 140. According to some embodiments, power source compartment 136 is configured to house at least one disposable power source, such as a battery. According to some embodiments, power source compartment 136 is configured to house at least one rechargeable power source, such as a rechargeable battery. According to some embodiments, power source compartment 136 comprises a power source.

According to some embodiment, aerosol generating device 100 further comprises a second pressure sensor 150, configured to detect the pressure in proximal compartment 104 and to generate signals indicative thereof. According to some embodiment, aerosol generating device 100 further comprises a second pressure sensor 150 configured to detect the pressure in proximal compartment 104 and to generate second pressure signals indicative thereof.

According to some embodiments, second pressure sensor 150 comprises a second differential pressure sensor. According to some embodiments, second pressure sensor 150 is positioned within proximal compartment 104. According to some embodiments, second pressure sensor 150 is attached to a sidewall of aerosol generating device 100. According to some embodiments, aerosol generating device 100 further comprises a measurement conduit (not numbered) open at one end to proximal compartment 104, and connected at the other end to second pressure sensor 150 (see FIG. 1). According to some embodiments, second pressure sensor 150 is configured to measure super atmospheric pressure. According to some embodiments, second pressure sensor 150 is configured to measure atmospheric and super atmospheric pressure. According to some embodiments, second pressure sensor 150 is configured to measure pressure in the range of 1 to 100 Bar.

According to some embodiments, CPU 134 is configured to receive additional signals from second pressure sensor 150, indicative of the pressure values measured by second pressure sensor 150. According to some embodiments, CPU 134 is configured to receive the second pressure signals from second pressure sensor 150. According to some embodiments, CPU 134 is configured to control operation of gas pump 140 as a function of the signals received from first pressure sensor 132 and second pressure sensor 150. According to some embodiments, CPU 134 is configured to control operation of gas pump 140 as a function of the second pressure signals.

According to some embodiments, second pressure sensor 150 is configured to detect the pressure drop across the at least one porous medium 108. The pressure drop across the at least one porous medium 108 may depend on the amount of fluid stored in the at least one porous medium 108. According to some embodiments, CPU 134 compares between pressure values detected by first pressure sensor 132 and second pressure sensor 150 to derive the pressure drop across the at least one porous medium 108.

According to some embodiments, aerosol generating device 100 further comprises a liquid reservoir 154, a liquid pump 156, and a liquid conduit 158. According to some embodiments, liquid is provided in liquid reservoir 154 for deliverance to the at least one porous medium 108. According to some embodiments, liquid pump 156 is configured to deliver liquid from liquid reservoir 154 to porous medium 108 via liquid conduit 158.

According to some embodiments, the amount of liquid transferred from liquid reservoir 154 to the at least one porous medium 108 is variable and occurs between successive inhalation events.

According to some embodiments, CPU 134 is configured to control operation of liquid pump 156. According to some embodiments, CPU 134 is configured to control operation of liquid pump 156 as a function of second pressure sensor 150. According to some embodiments, CPU 134 is configured to control operation of liquid pump 156 as a function of first pressure sensor 132 and second pressure sensor 150.

According to some embodiments, CPU 134 is configured to control operation of liquid pump 156 in response to the first pressure signals. According to some embodiments, CPU 134 is configured to control operation of liquid pump 156 in response to the second pressure signals. According to some embodiments, CPU 134 is configured to control operation of liquid pump 156 in response to the first pressure signals and the second pressure signals.

According to some embodiments, CPU 134 is configured to control operation of liquid pump 156 as a function of the first pressure signals. According to some embodiments, CPU 134 is further configured to determine whether the pressure in distal compartment 110 is higher or lower than a threshold pressure value, based on the first pressure signals, and is configured to activate liquid pump 156 when said pressure is lower than the pressure threshold value. According to some embodiments, the CPU 134 is further configured to deactivate liquid pump 156 when said pressure is higher than the pressure threshold value.

Distal compartment 110 is located between porous medium 108 and outlet 106, through which an aerosol generating device user inhales. As a result of an inhalation, CPU 134 activates gas pump 140, which ultimately results in aerosolization of liquid originally contained in porous medium 108. The aerosolization decreases the level of liquid contained in porous medium 108. The depletion process of liquid from medium 108 needs to be compensated by addition of liquid. Thus, upon the pressure inside distal compartment 110 being reduced, first pressure sensor 132 is configured to sense this pressure drop, and in response send corresponding first pressure signals to CPU 134, according to some embodiments. According to some embodiments, upon receiving the first pressure signals indicative of sub atmospheric pressure, CPU 134 acts to compensate for the subtracted amount of liquid by activating liquid pump 156 to deliver liquid from liquid reservoir 154 through liquid conduit 158 to porous medium 108. In order for to porous medium 108 not to be over-flooded the action of liquid pump 156 halts as detailed below, when referring to the second pressure signals.

According to some embodiments, controlling the operation of liquid pump 156 in response to the first pressure signals comprises controlling the rate of liquid delivery by liquid pump 156 to porous medium 108 based on the first pressure signals.

According to some embodiments, CPU 134 is configured to increase the rate of liquid delivery to porous medium 108 upon decrease of the pressure in distal compartment 110.

According to some embodiments, CPU 134 is configured variably to control operation of liquid pump 156 as a function of the second pressure signals.

According to some embodiments, CPU 134 is further configured to determine whether the pressure in proximal compartment 104 is higher or lower than a threshold pressure value, based on said second pressure signals, and is configured to activate liquid pump 156 when said pressure is lower than the pressure threshold value. According to some embodiments, CPU 134 is further configured to deactivate liquid pump 156 when said pressure is higher than the pressure threshold value.

Proximal compartment 104 is located between porous medium 108 and gas inlet 142, through which pressurized air from gas pump 140 enters. As a result of pressurized air entering proximal compartment 104, the pressure inside proximal compartment 104 is rises.

Without wishing to be bound by any theory or mechanism of action, upon operation of gas pump 140 the gas pressure inside proximal compartment 104 is positive (i.e. over 1 atmosphere). However, this positive pressure is not constant, and is a factor of the amount of liquid absorbed in porous medium 108. Specifically, when porous medium 108 is filled with liquid, its pores are occupied with molecules of the liquid and obstruct the passage of the pressurized air therethrough (i.e. from proximal compartment 104 to distal compartment 110), thereby increasing the positive pressure inside proximal compartment 104. In contrast, when porous medium 108 is empty or contains small amount of liquid, many of its pores are unoccupied and allow the passage of the pressurized air therethrough (i.e. from proximal compartment 104 to distal compartment 110), thereby decreasing the positive pressure inside proximal compartment 104.

Second pressure sensor 150 is configured to sense this increased or decreased pressure in proximal compartment 104, and in response send corresponding second pressure signals to CPU 134, according to some embodiments. According to some embodiments, upon receiving the second pressure signals indicative of the level of super atmospheric pressure, CPU 134 calculates the amount of liquid missing inside porous medium 108 to reach equilibrium level of liquid, according to some embodiments. According to some embodiments, according to said calculation CPU 134 controls the activation of liquid pump 156. In a first example, when the second pressure signals indicate to CPU 134 that the positive pressure is too high (e.g. above a predetermined pressure value) CPU 134 operates liquid pump 156 to halt the delivery of liquid form liquid reservoir 154 through liquid conduit 158 to porous medium 108. In a second example, when the second pressure signals indicate to CPU 134 that the positive pressure is substantially lower (e.g. substantially below a predetermined positive pressure value) CPU 134 operates liquid pump 156 to deliver substantial amounts of liquid form liquid reservoir 154 through liquid conduit 158 to porous medium 108. In a third example, when the second pressure signals indicate to CPU 134 that the positive pressure is moderately lower (e.g. a little below a predetermined positive pressure value) CPU 134 operates liquid pump 156 to deliver small volume of liquid form liquid reservoir 154 through liquid conduit 158 to porous medium 108. The above examples exemplify the variable nature of CPU 134 controlling liquid pump 156

According to some embodiments, controlling the operation of liquid pump 156 in response to the second pressure signals comprises controlling the rate of liquid delivery by liquid pump 156 to at least one porous medium 108 based on the second pressure signals.

According to some embodiments, 1 CPU 134 is configured to increase the rate of liquid delivery to porous medium 108 upon decrease of the pressure in proximal compartment 104.

According to some embodiments, CPU 134 is configured to analyze the rate of change of the pressure measured by second pressure sensor 150. According to some embodiments, CPU 134 is configured to variably control the operation of liquid pump 156 based on said analysis.

According to some embodiments, aerosol generating device 100 further comprises a second Pulse Wave Modulation (second PWM) component 152, such that the flow rate of liquid flowing through liquid pump 156 is adjusted via second PWM component 152 by CPU 134.

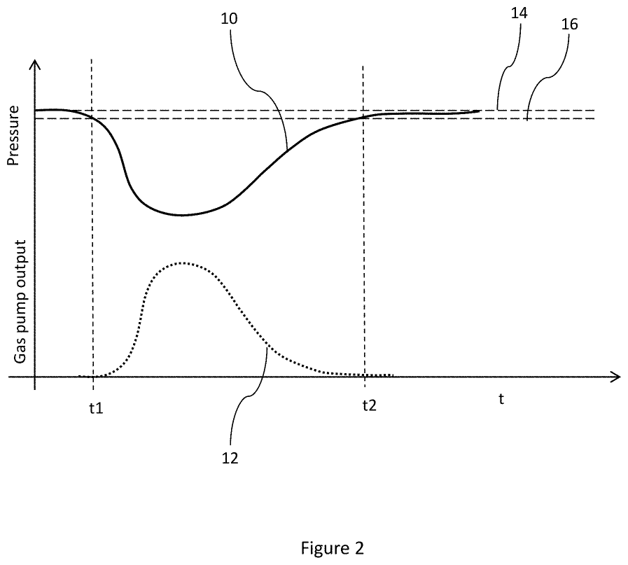

Reference is now made to FIG. 2. FIG. 2 depicts graphs of pressure and gas pump output as functions of time, according to some embodiments. Curve line 10 represents an exemplary varying pressure level in distal compartment 110 detected by first pressure sensor 132 during an inhalation maneuver. Pressure level 14 indicates ambient or atmospheric pressure. In the example of FIG. 2, the pressure indicated by curve line 10 dips from ambient pressure level 14 to a minimum pressure level (not numbered), and then rises again back to ambient pressure level 14. The waveform of curve line 10 is influenced, in some embodiments, by a user's inhalation maneuver. For example, a strong inhalation will result in a more significant reduction of pressure (and of by curve line 10) than a moderate inhalation.

According to some embodiments, a threshold pressure value 16 is predetermined, such that gas pump 140 is activated to operate only while the pressure detected by first pressure sensor 132 is lower than threshold pressure value 16. Curve line 12 indicates the output of gas pump 140 as a function of time. According to some embodiments, curve line 12 represents signal output, for example measured in voltage units of gas pump 140. According to some embodiments, curve line 12 represents gas flow from gas pump 140 flowing through gas inlet 142, for example in units of volumetric flow.

In the example depicted in FIG. 2, the pressure level detected by first pressure sensor 132 drops below threshold pressure value 16 at time t1. At that time, CPU 134 activates gas pump 140, either directly or via first PWM 138, to pump gas into gas inlet 142. When the pressure level rises above threshold pressure value 16 at time t2, CPU 134 stops activating gas pump 140.

According to some embodiments, CPU 134 is configured to detect whether pressure measured by first pressure sensor 132 is higher or lower than threshold pressure value 16.

According to some embodiments, the waveform of curve line 12 is in correlation with the waveform of curve line 10. According to some embodiments, the waveform of curve line 12 correlates with and follows the waveform of curve line 10. According to some embodiments, CPU 134 analyzes the rate of change of curve line 10, for example by deriving first and second derivatives thereof, to provide the signals to effect curve line 12.

Advantageously, delivery of aerosol from aerosol generating device 100 follows the intensity of inhalation, translated to the pressure level detected by first pressure sensor 132. For example, CPU 134 may calculate the area formed under the graph delimitated by curve line 10 and threshold pressure value 16, according to some embodiments. According to some embodiments, CPU 134 may operate gas pump 140 to generate pressurized air in an amount proportional to area under the graph. It is to be understood that the control of CPU 134 over the amount of pressurized air generated by gas pump 140 refers to the number of gas (e.g. air) molecules pressurized by the gas pump 140 during its action. This is a function, e.g. of the gas pressure, operation time and gas volume generated by gas pump 140 during its action.

A similar path may be employed with the control of CPU 134 over the operation of liquid pump 156. According to some embodiments, the waveform of curve line 12 is in correlation with a waveform of a curve line depicting the amount of liquid delivered by liquid pump 156. According to some embodiments, the waveform of curve line 12 correlates with and follows a waveform of a curve line depicting the amount of liquid delivered by liquid pump 156. According to some embodiments, CPU 134 analyzes the rate of change of curve line 10, for example by deriving first and second derivatives thereof, to provide the signals to a curve line depicting the amount of liquid delivered by liquid pump 156.

Advantageously, delivery of liquid from liquid reservoir 154 through liquid conduit 158 to porous medium 108 by liquid pump 156 follows the intensity of inhalation, translated to the pressure level detected by first pressure sensor 132. For example, CPU 134 may calculate the area formed under the graph delimitated by curve line 10 and threshold pressure value 16, according to some embodiments. According to some embodiments, CPU 134 may operate liquid pump 156 to deliver liquid from liquid reservoir 154 through liquid conduit 158 to porous medium 108 in an amount proportional to area under the graph. It is to be understood that the control of CPU 134 over the amount of liquid delivered by liquid pump 156 refers e.g. volume of liquid delivered by the by liquid pump 156 during its action. This is a function, e.g. of the liquid pressure and operation time of by liquid pump 156.

According to some embodiments, aerosol generating device 100 further comprises non-transient readable medium containing program instructions for CPU 134. According to some embodiments, the program instructions are configured to allow continuous monitoring of the pressure measured by first pressure sensor 132. According to some embodiments, the program instructions are configured to control either one of gas pump 140, first PWM 138, or both. According to some embodiments, the program instructions are configured to allow a set-up of threshold pressure value 16.

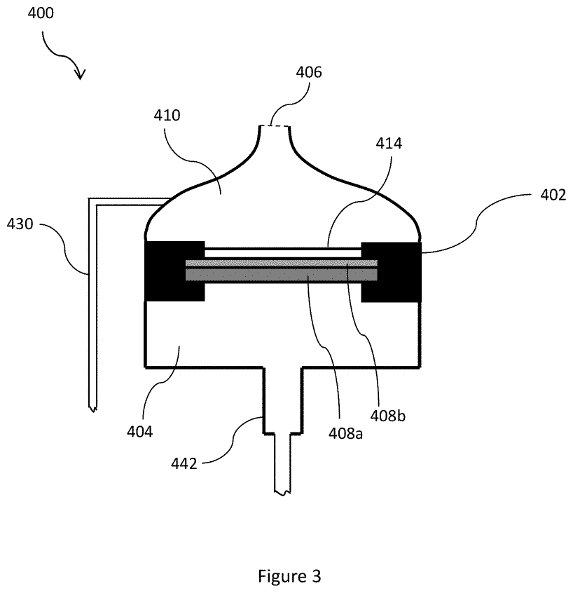

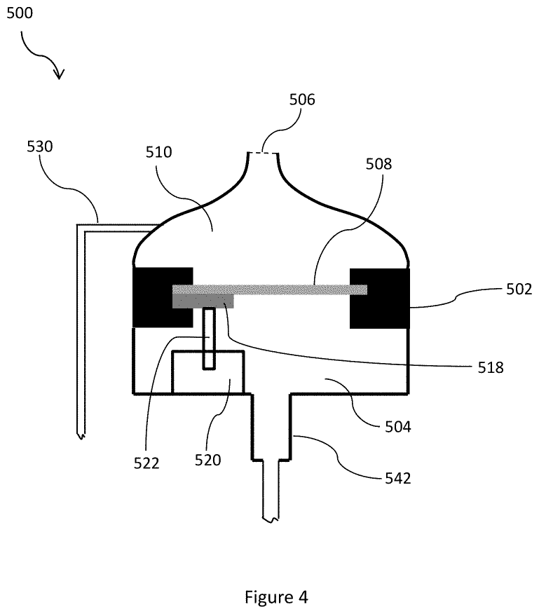

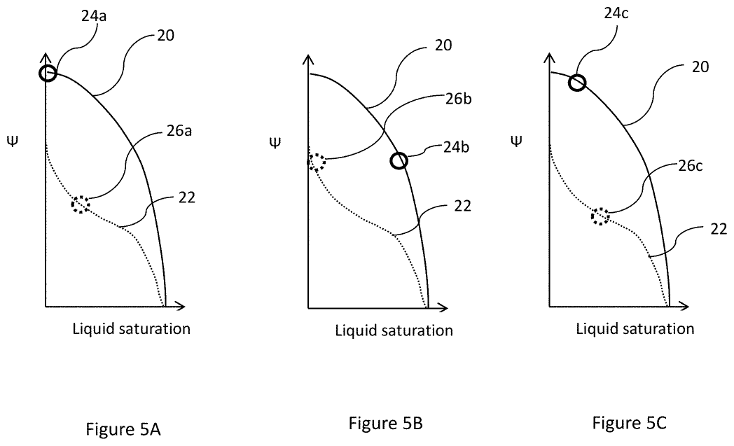

Reference is now made to FIG. 3. FIG. 3 constitutes a schematic partial illustration of aerosol generating device 400, according to some embodiments. Aerosol generating device 400 comprises at least one porous medium 408, a proximal compartment 404, a gas inlet 442, a distal compartment 410 and an outlet 406. According to some embodiments, the at least one porous medium 408 includes a plurality of porous media 408. In the example depicted in FIG. 4, the at least one porous medium 408 includes a proximal porous medium 408a and a distal porous medium 408b.

The term plurality, as used herein, refers to more than one or at least two.

According to some embodiments, each one of proximal porous medium 408a and distal porous medium 408b is similar in structure and functionality to the at least one porous medium 108.

According to some embodiments, proximal compartment 404, gas inlet 442, distal compartment 410 and outlet 406 are similar in structure and functionality to proximal compartment 104, gas inlet 142, distal compartment 110 and outlet 106, respectively.

According to some embodiments, proximal porous medium 408a and distal porous medium 408b are pressed against each other. According to some embodiments, proximal porous medium 408a and distal porous medium 408b are in contact with one another, configured to allow fluid flow there between. According to some embodiments, proximal porous medium 408a and distal porous medium 408b are in contact with one another, configured to allow fluid flow there between and there through. In the example depicted in FIG. 3, the distal side of proximal porous medium 408a is in contact with the proximal side of distal porous medium 408b.

According to some embodiments, proximal porous medium 408a is characterized by higher porosity than distal porous medium 408b. According to some embodiments, the pores of proximal porous medium 408a are larger than the pores of distal porous medium 408b. According to some embodiments, the distribution of pores of proximal porous medium 408a is more dense than the distribution of pores of distal porous medium 408b. According to some embodiments, proximal porous medium 408a is characterized by a lower Laplace pressure than that of distal porous medium 408b.

According to some embodiments, proximal porous medium 408a comprises a plurality of pores having first average diameter and distal porous medium 408b comprises a plurality of pores having second average diameter, wherein the first average diameter is larger than the second average diameter by at least 5%. According to some embodiments, the ratio the first average diameter is larger than the second average diameter by at least 10%. According to some embodiments, the ratio the first average diameter is larger than the second average diameter by at least 15%. According to some embodiments, the ratio the first average diameter is larger than the second average diameter by at least 20%. According to some embodiments, the ratio the first average diameter is larger than the second average diameter by at least 25%. According to some embodiments, the ratio the first average diameter is larger than the second average diameter by at least 40%. According to some embodiments, the ratio the first average diameter is larger than the second average diameter by at least 50%. According to some embodiments, the ratio the first average diameter is larger than the second average diameter by at least 75%. According to some embodiments, the ratio the first average diameter is larger than the second average diameter by at least 90%.

According to some embodiments, proximal porous medium 408a comprises a plurality of pores having first average diameter and distal porous medium 408b comprises a plurality of pores having second average diameter, wherein the ratio between the first average diameter and the second average diameter is at least 2:1. According to some embodiments, the ratio is at least 4:1. According to some embodiments, the ratio is at least 7:1. According to some embodiments, the ratio is at least 10:1. According to some embodiments, the ratio is at least 20:1. According to some embodiments, the ratio is at least 20:1. According to some embodiments, the ratio is at least 50:1. According to some embodiments, the ratio is at least 100:1.

According to some embodiments, the second average diameter is in the range of 1-3 μm. According to some embodiments, the second average diameter is in the range of 1.5-2.5 μm. According to some embodiments, the second average diameter is in the range of 1.8-2.2 μm. According to some embodiments, the first average diameter is in the range of 3-300 μm. According to some embodiments, the second average diameter is in the range of 5-100 μm. According to some embodiments, the second average diameter is in the range of 10-100 μm.

According to some embodiments, proximal porous medium 408a comprises a first porosity and distal porous medium 408b comprises a second porosity, wherein the first porosity is larger than the second porosity by at least 5%. It is to be understood that porosity of porous media is measured as percentage open space of the media. For example, in case that a medium A is characterized by having 10% of it volume as open space, its porosity is defined as 10%. Said 10% refers to volume of medium A, which is unoccupied by the medium material. Thus, 90% of medium A comprises the medium material. In addition, in case that a medium B has 20% porosity, the ratio between the porosity of medium B and the porosity of medium A is 2:1, and the porosity of medium B is larger than porosity of medium A by 100%.

According to some embodiments, the first porosity is larger than the second porosity by at least 10%. According to some embodiments, the first porosity is larger than the second porosity by at least 20%. According to some embodiments, the first porosity is larger than the second porosity by at least 30%. According to some embodiments, the first porosity is larger than the second porosity by at least 40%. According to some embodiments, the first porosity is larger than the second porosity by at least 50%. According to some embodiments, the first porosity is larger than the second porosity by at least 60%. According to some embodiments, the first porosity is larger than the second porosity by at least 70%. According to some embodiments, the first porosity is larger than the second porosity by at least 80%. According to some embodiments, the first porosity is larger than the second porosity by at least 90%. According to some embodiments, the first porosity is larger than the second porosity by at least 100%. According to some embodiments, the first porosity is larger than the second porosity by at least 150%. According to some embodiments, the first porosity is larger than the second porosity by at least 200%. According to some embodiments, the first porosity is larger than the second porosity by at least 150%. According to some embodiments, the first porosity is larger than the second porosity by at least 300%. According to some embodiments, the first porosity is larger than the second porosity by at least 350%. According to some embodiments, the first porosity is larger than the second porosity by at least 400%. According to some embodiments, the first porosity is larger than the second porosity by at least 450%. According to some embodiments, the first porosity is larger than the second porosity by at least 500%. According to some embodiments, the first porosity is larger than the second porosity by at least 550%. According to some embodiments, the first porosity is larger than the second porosity by at least 600%.

According to some embodiments, the second porosity is in the range of 5-30%. According to some embodiments, the second porosity is in the range of 5-20%. According to some embodiments, the second porosity is in the range of 5-15%. According to some embodiments, the second porosity is in the range of 7-13%. According to some embodiments, the second porosity is in the range of 8-12%.

According to some embodiments, the first porosity is in the range of 15-90%. According to some embodiments, the first porosity is in the range of 20-80%. According to some embodiments, the first porosity is in the range of 20-70%. According to some embodiments, the first porosity is in the range of 20-50%.

According to some embodiments, proximal porous medium 408a is spaced from distal porous medium 408b such that in use, liquid partitions between porous mediums 408a and 408b, so as to establish equilibrium governed by capillary forces in each of proximal porous medium 408a and distal porous medium 408b.

In use, the amount of liquid in distal porous medium 408b during aerosolization decreases and its matric potential becomes more negative, which results in transfer of liquid from proximal porous medium 408a thereto.

According to some embodiments, aerosol generating device 400 further comprises a filter 414. According to some embodiments, filter 414 is configured to filter the passage of droplets depending on their diameter, such that large diameter droplets are obstructed thereby.

According to some embodiments, the at least one porous medium 108 is configured to act as an impactor. According to some embodiments, the at least one porous medium 108 is an impactor. According to some embodiments, the at least one porous medium 108 is configured to act as a filter. According to some embodiments, at least one porous medium 108 material is the filter. According to some embodiments, the impactor is an independent structure, different from the at least one porous medium 108. According to some embodiments, filter 414 is an independent structure, different from the at least one porous medium 108.

Without being bound by any theory or mechanism of action, control over droplet size of generated aerosol is achieved by including the impactor or the filter in the aerosol generating device described herein. Thus, according to some embodiments, control over droplet size of generated aerosol is achieved by introducing filter 414.

According to some embodiments, filter 414 comprises a porous medium. According to some embodiments, filter 414 comprises foam. According to some embodiments, filter 414 comprises a hydrophobic foam. According to some embodiments, filter 414 is consisting of foam, such as, a hydrophobic foam. According to some embodiments, filter 414 is configured to allow passage of fluid there through without retaining the fluid therein, thereby allowing the fluid to imbibe back into the at least one porous medium 108 when pressure drop across the at least one porous medium 108 is drops below a predefined value.

According to some embodiments, aerosol generating device 400 further comprises a support 402, similar in structure and functionality to support 102. According to some embodiments, each one of proximal porous medium 408a and distal porous medium 408b is attached to support 402, such as to prevent displacements in the distal or proximal directions of each or avoid unintentional displacements in the distal or proximal directions of each.

According to some embodiments, aerosol generating device 400 further comprises a gas pump, a first pressure sensor and a CPU, similar in structure and functionality to gas pump 140, first pressure sensor 132 and CPU 134, respectively.

According to some embodiments, aerosol generating device 400 further comprises a measurement conduit 430, similar in structure and functionality to measurement conduit 130.

According to some embodiments, aerosol generating device 400 further comprises a first PWM, similar in structure and functionality to first PWM 138.

According to some embodiments, aerosol generating device 400 further comprises a power source compartment, similar in structure and functionality to power source compartment 136.

According to some embodiments, aerosol generating device 400 further comprises a second pressure sensor, similar in structure and functionality to second pressure sensor 150.

According to some embodiments, aerosol generating device 400 further comprises a liquid reservoir, a liquid pump, and a liquid conduit, similar in structure and functionality to liquid reservoir 154, liquid pump 156, and liquid conduit 158.

According to some embodiments, aerosol generating device 400 further comprises a second PWM, similar in structure and functionality to second PWM 152.

Reference is now made to FIG. 4. FIG. 4 constitutes a schematic partial illustration of aerosol generating device 500, according to some embodiments. Aerosol generating device 500 comprises at least one porous medium 508, a proximal compartment 504, a gas inlet 542, a distal compartment 510 and an outlet 506. According to some embodiments, at least one porous medium 508, proximal compartment 504, gas inlet 542, distal compartment 510 and outlet 506 are similar in structure and functionality to at least one porous medium 108, proximal compartment 104, gas inlet 142, distal compartment 110 and outlet 106, respectively.

According to some embodiments, aerosol generating device 500 further comprises a liquid container 520 and a liquid drawing element 522. According to some embodiments, liquid is provided in liquid container 520 for deliverance towards the at least one porous medium 508 via liquid drawing element 522. According to some embodiments, the liquid is similar in properties to the liquid described with respect to aerosol generating device 100.

According to some embodiments, aerosol generating device 500 comprises a single liquid drawing element 522.

According to some embodiments, liquid drawing element 522 is configured to absorb liquid in an amount which is at least 100% of its weight. According to some embodiments, liquid drawing element 522 is configured to absorb liquid in an amount which is at least 150% of its weight. According to some embodiments, the at least one stationary liquid absorbing element is configured to absorb liquid in an amount which is at least 200% of its weight.

According to some embodiments, liquid drawing element 522 comprises cloth, wool, felt, sponge, foam, cellulose, yarn, microfiber or a combination thereof, having high tendency to absorb aqueous solutions. Each possibility represents a separate embodiment. According to some embodiments, the sponge is an open cell sponge. According to some embodiments, the sponge is a closed cell sponge.

According to some embodiments, liquid drawing element 522 comprises a capillary valve, configured to allow fluid passage there through in a direction from the proximal end to the distal end thereof, while preventing gas or air flow in the opposite direction.

According to some embodiments, liquid drawing element 522 comprises fabric. Specifically, fibrous and/or woven fabric, such as a wick, is a hydrophilic and liquid absorbing material, which may be used as the stationary liquid absorbing element(s), according to some embodiments.

According to some embodiments, liquid drawing element 522 is a hydrophilic liquid drawing element. According to some embodiments, liquid drawing element 522 is a hydrophilic sponge.

According to some embodiments, liquid drawing element 522 is pressed against the at least one porous medium 508. According to some embodiments, liquid drawing element 522 is in contact with the at least one porous medium 508, configured to allow fluid flow there between. According to some embodiments, the proximal side of the at least one porous medium 508 is in contact with a distal end of liquid drawing element 522, wherein a proximal end of liquid drawing element 522 is dipped within liquid container 520. According to some embodiments, liquid drawing element 522 includes barrier layers, configured to allow fluid passage there through in a direction from the proximal end to the distal end thereof, while preventing gas or air flow in the opposite direction. According to some embodiments, liquid drawing element 522 is configured to discharge at least portions of the liquid absorbed therein into at least some of the plurality of pores of the at least one porous medium 508.

Without wishing to be bound by any theory or mechanism of action, when liquid drawing element 522 comprises a hydrophilic sponge, at it comes in contact with the liquid in liquid container 520, capillary action within and among the pores of the sponge lead to it being absorbed.

According to some embodiments, aerosol generating device 500 further comprises liquid-gating porous medium 518. According to some embodiments, liquid-gating porous medium 518 is similar in properties and function to proximal porous medium 408a.

According to some embodiments, liquid-gating porous medium 518 comprises a plurality of pores having first average diameter and at least one porous medium 508 comprises a plurality of pores having second average diameter, wherein the ratio between the first average diameter and the second average diameter is at least 2:1. According to some embodiments, the ratio is at least 4:1. According to some embodiments, the ratio is at least 7:1. According to some embodiments, the ratio is at least 10:1. According to some embodiments, the ratio is at least 20:1. According to some embodiments, the ratio is at least 20:1. According to some embodiments, the ration is at least 50:1. According to some embodiments, the ratio is at least 100:1.

According to some embodiments, liquid-gating porous medium 518 is characterized by higher porosity than that of the at least one porous medium 508.

According to some embodiments, the pores of liquid-gating porous medium 518 are larger than the pores of the at least one porous medium 508.

According to some embodiments, liquid-gating porous medium 518 is pressed against the at least one porous medium 508. According to some embodiments, liquid-gating porous medium 518 is in contact with the at least one porous medium 508, configured to allow fluid flow there between. According to some embodiments, the proximal side of liquid-gating porous medium 518 is in contact with the distal end of liquid drawing element 522, wherein the proximal end of liquid drawing element 522 is dipped within liquid container 520.

According to some embodiments, liquid drawing element 522 is configured to maintain at least one of liquid-gating porous medium 518 or porous medium 508 between predefined matric potential values. According to some embodiments, liquid drawing element 522 is configured to transfer liquid from liquid container 520 towards the at least one porous medium 508 only upon reaching a matric potential value which is more negative than a predefined matric potential threshold. According to some embodiments, liquid drawing element 522 is configured to discharge at least portions of the liquid absorbed therein into at least some of the plurality of pores of liquid-gating porous medium 518.

According to some embodiments, liquid-gating porous medium 518 is spaced from the at least one porous medium 508 such that in use, liquid partitions liquid-gating porous medium 518 and porous medium 508 so as to establish equilibrium governed by capillary forces therein.

In use, the amount of liquid in the at least one porous medium 508 during aerosolization decreases and its matric potential becomes more negative, which results in transfer of liquid from liquid-gating porous medium 518 thereto. In turn, liquid-gating porous medium 518 pulls additional liquid from liquid container 520 via liquid drawing element 522.

According to some embodiments, aerosol generating device 500 further comprises a support 502, similar in structure and functionality to support 502. According to some embodiments, liquid-gating porous medium 518 is attached to at least a portion of support 502, such as to prevent displacements in the distal or proximal directions thereof

According to some embodiments, aerosol generating device 500 further comprises a gas pump 540, a first pressure sensor 532 and a CPU 534, similar in structure and functionality to gas pump 540, first pressure sensor 532 and CPU 534, respectively.

According to some embodiments, aerosol generating device 500 further comprises a measurement conduit 530, similar in structure and functionality to measurement conduit 130.

According to some embodiments, aerosol generating device 500 further comprises a first PWM 538, similar in structure and functionality to first PWM 138.

According to some embodiments, aerosol generating device 500 further comprises a power source compartment 536, similar in structure and functionality to power source compartment 136.

According to some embodiments, aerosol generating device 500 further comprises a second pressure sensor 550, similar in structure and functionality to second pressure sensor 150.

According to some embodiments, aerosol generating device 500 further comprises a liquid reservoir 554, a liquid pump 556, and a liquid conduit 558, similar in structure and functionality to liquid reservoir 154, liquid pump 156, and liquid conduit 158.

According to some embodiments, aerosol generating device 500 further comprises a second PWM 552, similar in structure and functionality to second PWM 152.

According to some embodiments, the liquid contained within either liquid reservoir 154, 454, 554 or liquid container 520 is saline, water, carrier, cleansing liquid and the like.

Reference is now made to FIG. 6A-C. FIG. 6A-C depicts matric potential vs. liquid saturation functions, according to some embodiments. Curves 20 and 22 represent matric potential curves of a distal porous medium and a proximal porous medium, respectively. According to some embodiments, the distal porous medium is distal porous medium 408b and the proximal porous medium is proximal porous medium 408a According to some embodiments, the distal porous medium is porous medium 508 and the proximal porous medium is liquid-gating porous medium 518.

Matric potential values 24a, 24b and 24c represent the matric potential of the distal porous medium, in different scenarios depicted in FIGS. 6A 6B and 6C, respectively. Matric potential values 26a, 26b and 26c represent the matric potential of the proximal porous medium in the scenarios depicted in FIGS. 6A 6B and 6C, respectively

FIG. 6A depicts a hypothetical scenario in which the distal porous medium is depleted of fluid, while the proximal porous medium is partially filled. FIG. 6B depicts a scenario in which the distal and the proximal porous mediums contact each other, such that the distal porous medium is partially saturated due to fluid entering thereto from the proximal porous medium, resulting in a matric potential 24b becoming less negative than matric potential 24a, while the matric level 26b is more negative than 26a due to liquid depletion from the proximal porous medium. Since liquid is transferred from the proximal porous medium to distal the porous medium, liquid movement stops when both distal and proximal porous mediums are in equilibrium.

FIG. 6C depicts a scenario in which liquid is depleted from the distal porous medium due to aerosolization, resulting in matric potential 24c becoming more negative than 24b. The proximal porous medium has been replenished with liquid, either from liquid reservoir 154 or from a liquid container 520, resulting in matric potential 26c becoming less negative than 26b.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

1.-33. (canceled)

34. An aerosol generating device comprising:
at least one porous medium;
a gas inlet;
a proximal compartment between the gas inlet and the at least one porous medium;
an outlet;
a distal compartment between the at least one porous medium and the outlet;
a first pressure sensor configured to detect the pressure in the distal compartment and to generate first pressure signals indicative thereof a second pressure sensor configured to detect the pressure in the proximal compartment and to generate second pressure signals indicative thereof;
a gas pump configured to deliver compressed gas via the gas inlet, through the proximal compartment, towards the at least one porous medium;
a liquid pump configured to deliver liquid from a liquid reservoir to the at least one porous media, through a liquid conduit; and
a Central Processing Unit (CPU) configured to receive the first pressure signals from the first pressure sensor and the second pressure signals from the second pressure sensor, wherein the CPU is further configured to control operation of the gas pump in response to the first pressure signals; and configured to control operation of the liquid pump in response to the first pressure signals and the second pressure signals.

35. The aerosol generating device of claim 34, further comprising a first Pulse Wave Modulation component, controlled by the CPU and configured to adjust the flow rate of gas from the gas pump.

36. The aerosol generating device of claim 34, further comprising a measurement conduit, open at one end thereof to the distal compartment, and connected at the other end thereof to the first pressure sensor.

37. The aerosol generating device of claim 34, wherein the CPU is configured to variably control operation of the gas pump as a function of the first pressure signals.

38. The aerosol generating device of claim 34, wherein the CPU is further configured to determine whether the pressure in the distal compartment is higher or lower than a threshold pressure value, based on said first pressure signals, and is configured to activate the gas pump when said pressure is lower than the pressure threshold value.

39. The aerosol generating device of claim 38, wherein the CPU is further configured to deactivate the gas pump when said pressure is higher than the pressure threshold value.

40. The aerosol generating device of claim 34, wherein the CPU is configured to vary the power supplied to the gas pump, wherein the gas pump is configured to deliver compressed gas at variable compression levels in response to the varying power supplied thereto.

41. The aerosol generating device of claim 40, wherein the CPU is configured to increase the power supplied to the gas pump upon decrease of the pressure in the distal compartment.

42. The aerosol generating device of claim 34, wherein the CPU is configured to analyze the rate of change of the pressure measured by the first pressure sensor, wherein the CPU is further configured to analyze the rate of change of the pressure measured by the second pressure sensor.

43. The aerosol generating device of claim 34, wherein the CPU is configured to variably control operation of the liquid pump as a function of the first pressure signals.

44. The aerosol generating device of claim 34, wherein the CPU is further configured to determine whether the pressure in the distal compartment is higher or lower than a threshold pressure value, based on said first pressure signals, and is configured to activate the liquid pump when said pressure is lower than the pressure threshold value.

45. The aerosol generating device of claim 34, wherein controlling the operation of the liquid pump in response to the first pressure signals comprises controlling the rate of liquid delivery by the liquid pump to the at least one porous medium based on the first pressure signals.

46. The aerosol generating device of claim 34, wherein the CPU is configured to increase the rate of liquid delivery to the porous medium upon decrease of the pressure in the distal compartment.

47. The aerosol generating device of claim 34, wherein the CPU is further configured to determine whether the pressure in the proximal compartment is higher or lower than a threshold pressure value, based on said second pressure signals, and is configured to activate the liquid pump when said pressure is lower than the pressure threshold value.

48. The aerosol generating device of claim 34, wherein the CPU is configured to increase the rate of liquid delivery to the porous medium upon decrease of the pressure in the proximal compartment.

49. An aerosol generating device comprising:
at least one proximal porous medium;
at least one distal porous medium;
a gas inlet;
a proximal compartment between the gas inlet and the at least one proximal porous medium;
an outlet;
a distal compartment between the at least one distal porous medium and the outlet;
a first pressure sensor configured to detect the pressure in the distal compartment and to generate first pressure signals indicative thereof;
a gas pump configured to deliver compressed gas via the gas inlet, through the proximal compartment, towards the at least one proximal porous medium; and
a Central Processing Unit (CPU) configured to receive the first pressure signals from the first pressure sensor and configured to control operation of the gas pump in response to the first pressure signals.
wherein the proximal porous medium is in contact with the distal porous medium; and
wherein the proximal porous medium is characterized with higher porosity than the distal porous medium.

50. The aerosol generating device of claim 46, wherein the CPU is configured to variably control operation of the gas pump as a function of the signals received from the first pressure sensor.

51. The aerosol generating device of claim 34, further comprising a filter configured to filter the passage of droplets depending on their diameter, such that large diameter droplets are obstructed thereby; wherein the filter is in contact with the distal porous medium.

52. The aerosol generating device of claim 46, further comprising a liquid container and a liquid drawing element dipped within the liquid container, configured to deliver liquid from the liquid container towards the at least one distal porous medium.

53. The aerosol generating device claim 53, wherein the liquid drawing element is in contact with the at least one distal porous medium.
